(19) United States
(12) Patent Application Publication
(10) Pub. No.: US 2005/0090778 A1
(43) Pub. Date: Apr. 28, 2005

(54) Apparatus for the microscopically-dosed injection of an active product by means of jets of pressurized working liquid and the method of generating a sequence of liquid jets using said apparatus

(76) Inventor: Bertrand Gonon, Ternay (FR)

Correspondence Address: Davis and Bujold, Fourth Floor, 500 N Commercial Street, Manchester, NH, 03101, US

(21) Appl. No.: 10/500,990

(22) PCT Filed: Jan. 7, 2003
(86) PCT No.: PCT/FR03/00026

(30) Foreign Application Priority Data
Jan. 7, 2002 (FR) 02/00129

(51) Int. Cl. A61M 37/00

(52) U.S. Cl. 604/140

(57) ABSTRACT

The invention relates to an injection apparatus (1) which blasts pressurised working liquid against the tissue to be treated in order to hollow out an injection channel and which subsequently injects the active product into said channel. The inventive apparatus comprises a reserve of working liquid (2) which can be pressurised and which is connected to a handpiece (5) by means of a main branch (4) of the fluid system. An active end piece (8) is disposed at the end of the handpiece comprising an outlet for a pressurised jet of working liquid. Moreover, the invention comprises a secondary branch opening (25) which is parallel to the main branch and which is fluidically isolated from same. The secondary branch is intended to contain the active product. According to each of the two branches of the system according to pre-determined parameters.

The present invention concerns a surgical, medical or diagnostic apparatus using jets of pressurized liquid to achieve one or more microscopically-dosed injections of an active or treatment product. It also concerns an injection method utilizing this apparatus.

More generally, it also concerns a method using an apparatus to generate a sequence of liquid jets comprising at least one impulse of a pressurized working liquid followed by one microscopically dosed impulse of an active product.

In the realm of medical treatment, during a surgical intervention or a diagnostic procedure, it is often advantageous to administer an active product such as a medication, a product used in surgical intervention, or a diagnostic agent by placing it in direct contact with the injured area or the area to be treated, usually located inside an organ or the tissue of a human body.

The conventional method of doing this is to use an injection catheter introduced and moved along a vessel inside the patient's body until its distal extremity, which may have one or more orifices or which may extend into an injection needle, is located as close at possible to the area to be treated. Then the pressurized active product is injected onto the area to be treated through the one or more orifices or the needle tip of the injection catheter.

Current developments in gene and cell therapy require the use of specific active products such as cells, genes, plasmids or proteins, for example, which are delicate to inject, raising new problems.

These products, often having a live cellular base, are particularly fragile. For example, they are sensitive to shock, excessive pressure elevation, and to narrow diameter conduits. Moreover, they must be injected in very small quantities while still maintaining precise dilution and dosage.

Furthermore, these products diffuse poorly. When injected using a conventional syringe or needle injection catheter, rather than diffusing progressively, these products tend to remain in a localized sphere near the point of injection. The effectiveness of treatment is greatly reduced and multiple injection points must be used.

The goal of the invention is to provide an improved apparatus and method of injection specifically for this type of product.

Devices without needles for injecting pressurized treatment products are already known in the art. When using this type of device, the tissue is no longer mechanically punctured prior to injection. Penetration through the tissue is accomplished by the pressurized force of the liquid which, after ejection, strikes the tissue with enough force to puncture it and form an injection channel, then diffuses throughout the tissue using this channel.

Due to the pressure with which it is injected, the treatment liquid penetrates much more deeply than with a conventional needle injection device and diffusion is more extensive.

These devices may be equipped with a catheter to allow injection inside the patient's body, directly contacting the organ or tissue to be treated.

However, while these pressure injection devices offer many advantages over conventional devices, they cannot be adapted to inject the active products used in the fields of gene therapy or cell therapy. These products, which often contain viruses or live cells, are actually very fragile. When injected using this type of pressure device the products deteriorate from the violent force of striking the tissue to be treated, generally causing the live cells to die and destroying the product's potential for activity.

The goal of this invention is to teach the use of a pressure injection apparatus offering all the advantages of these types of devices, but which also allows microscopically dosed injection of a fragile, active product such as a transgenetic product, for example, without any deterioration.

The inventive principle consists of dissociating the phase of forming an injection channel, which is harmful to fragile substances, from the phase of injecting an active product.

The device according to the invention blasts the pressurized working liquid onto the tissue to be treated so that it forms a hollow injection channel, and then injects the active product into the channel.

These two operations occur successively but within a brief time period and using the same device. The surgeon can blast the active product several times in succession, thus injecting it several times, either in the same injection channel or at several locations on the tissue to be treated, with the parameters for each blasting operation in the injection process having been previously selected and programmed by the surgeon so as to optimize injection for each individual case.

Since the active product is not used to form a hollow conduit, it is not subjected to violent shock and it is perfectly preserved. However, it may be propelled under pressure deeply within the tissue to be treated. Therefore, diffusion is greatly improved in relation to a conventional injection apparatus.

Patent Application WO 00/56232 of SAPHIR MEDICAL PRODUCTS GMBH discloses a dissection apparatus using pressurized liquid which also allows the injection of treatment liquid using pressurized projection.

This device provides for either directly mixing treatment product in the reserve of working liquid, or for the provision of a reserve of working liquid and an independent reserve of treatment product, both of which are pressurized by a pressure generator.

In the first instance the active product is diluted in the working liquid and used simultaneously with it. This arrangement would not be suitable for the fragile products that are the subject of this invention. They would actually be destroyed while forming the injection channel.

Furthermore, the working liquid, usually physiological serum, is usually packaged in flexible plastic pouches containing a large volume, generally a liter. The active product, which is expensive and difficult to produce, is generally provided in small quantities and would become highly diluted in the reserve of working liquid, causing the liquid to be injected in a concentration that would incompatible with the application sought.

In the second instance, a reserve of treatment product is pressurized by the pressurized liquid generator. The surgeon releases the dose of active product using a switch or mixing device that are not shown.

Application No. WO 00/56232 also discloses the general concept of combining impulses of working liquid and treatment product within a series of discontinuous impulses constituting a pulsating spray using a multiplexing device that is not described.

These different variations are depicted very generally without any description of precise embodiments, and thus they remain in a purely theoretical plane.

The invention turns this general principle a into a concrete realization by teaching a derivation device that plays the role of a mixing, switching, or multiplexing device.

The injection apparatus of the invention comprises, as with the apparatus described in this prior art application, a reserve of working liquid that can be pressurized by a pressurized liquid generator, a handpiece terminating in an active extremity comprising outlets for the pressurized jets, either continuous or pulsating, of working liquid, and a jet of active product.

According to the invention, the apparatus further comprises:
a principal fluid circuit branch connecting the reserve of working liquid to the handpiece, with the flow of liquid through it being controlled by a first valve;
a second derivation branch, parallel to the principal branch and fluidly insulated from it, designed to contain the active product, with the flow of liquid through it being controlled by a second valve; and
a multiplexing means for independently controlling the opening and closing of the valves on each of the two circuit branches according to predetermined parameters.

These parameters may vary and have been previously selected and stored by the surgeon depending upon the particular case to be treated.

Varying the parameters for opening the two valves on the two circuit branches opens numerous injection possibilities to the surgeon using the apparatus of the invention.

Thus, for example, the invention teaches a method for generating a series of liquid jets using a similar apparatus in which the two valves on the two circuit branches open simultaneously to produce this mixture in the active extremity of the apparatus and for generating a jet consisting of a mixture of active product and working liquid in proportions that are precisely fixed by regulating the opening time of each of these valves.

The invention also teaches a method of injection using this device in which an injection channel is hollowed out by blasting a jet of pressurized working liquid and then, in a later phase, injecting the active product through the injection channel.

Since the injection channel has been initially formed by blasting a jet of pressurized working liquid, the active product injected during the second phase is preserved perfectly.

More generally, the invention teaches a method of generating a sequence of liquid jets using an apparatus comprising a reserve of working liquid pressurized by a pressurized liquid generator, a handpiece terminating in an active extremity comprising outlets for a jet of pressurized working liquid and a jet of active product, a principal fluid circuit branch connecting the reserve of working liquid to the handpiece, with the flow of liquid controlled by opening or closing an isolation valve, a secondary derivation branch parallel to the principal branch and fluidly isolated from it, designed to contain an active product and whose flow is controlled by opening or closing an isolation valve, and a multiplexing means to independently control the opening and closing of the isolation valves according to predetermined parameters. This method is characterized by generating a sequence comprising at least one impulse of pressurized working liquid, followed by a microscopically dosed impulse of active product.

According to this method, first the valve on the main circuit branch is opened to generate an impulse formed of an appropriate quantity of pressurized working liquid, and then in the next step the valve on the derivation branch is opened to generate an impulse formed of the desired quantity of active product.

Here again, the quantities of liquid that form the impulses of working liquid and active product can be established precisely by fixing the time for opening each of the valves.

The impulse of active product may possibly be followed by another impulse of pressurized working liquid. This second impulse may serve to push the active product deeper into the core of the tissue to be treated.

The impulse of active product may also be generated under high pressure, that is, pressure that essentially corresponds to the pressure of the working liquid.

However, certain especially fragile products cannot withstand being submitted to such pressure. In a particularly advantageous manner, the impulse of active product may also be generated at less pressure than that of the working product and preferably at low pressure.

Because of this inventive concept, the apparatus and the method of to the invention both allow the use of very small quantities of active product and permit microscopic doses of the product to be administered.

The apparatus and the method of the invention are therefore very well suited to the injection of active transgenetic products. However, they may also be used in all sorts of other surgical, medical, therapeutic and diagnostic applications, as well as applications relating to different technical domains.

Other characteristics and features of the invention will be apparent from reading the following detailed description in conjunction with the attached drawings, wherein:

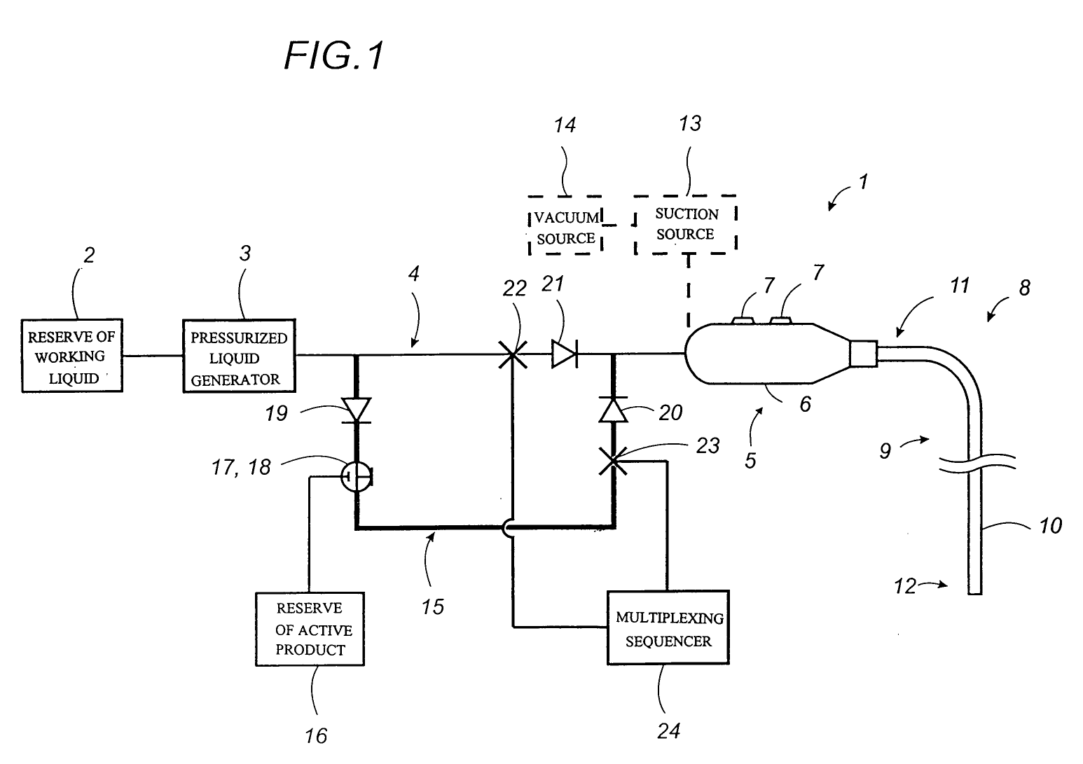

FIG. 1 is a general schematic view of the injection apparatus of the invention;

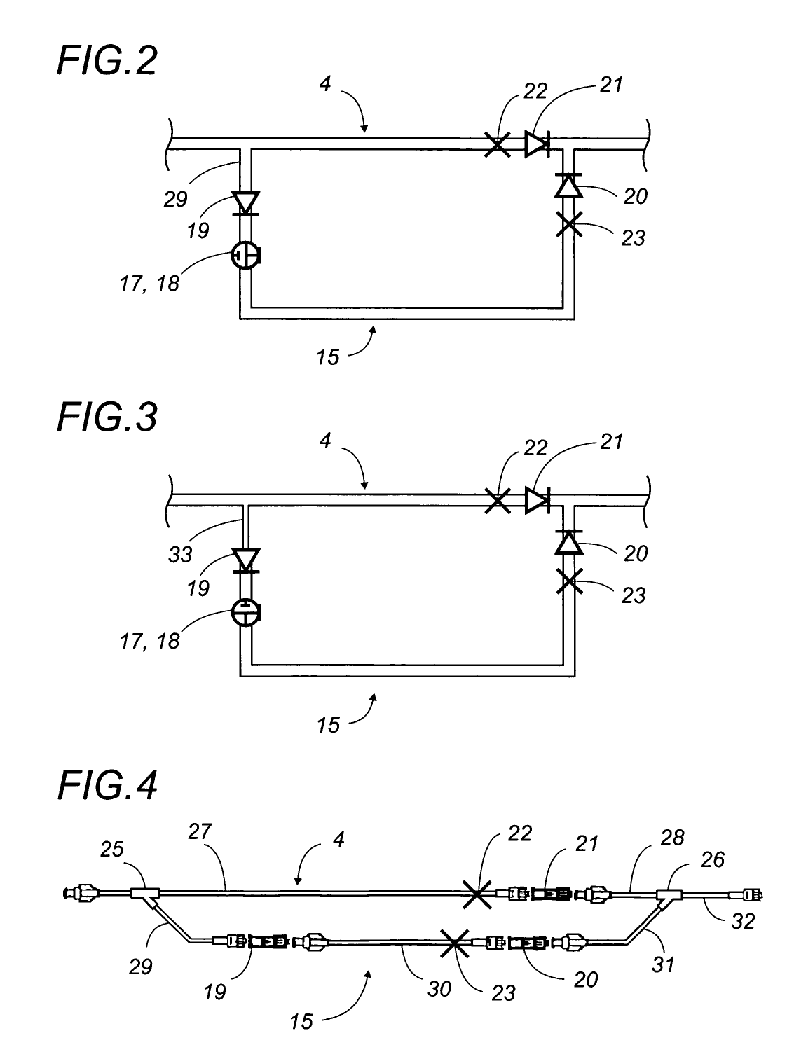

FIG. 2 is a schematic view of a portion of the injection apparatus according to the invention that corresponds to the principal branch and the derivation branch of the hydraulic circuit;

FIG. 3 is a schematic view similar to FIG. 2 showing the portion of the injection apparatus of the invention that corresponds to the principal branch and the derivation branch of the circuit with a narrow diameter conduit;

FIG. 4 is a view of another variation of the portion of the injection apparatus of the invention that corresponds to the principal branch and the derivation branch of the circuit, without any valve for introducing active product, but with the derivation tube serving as a measured reserve of active product;

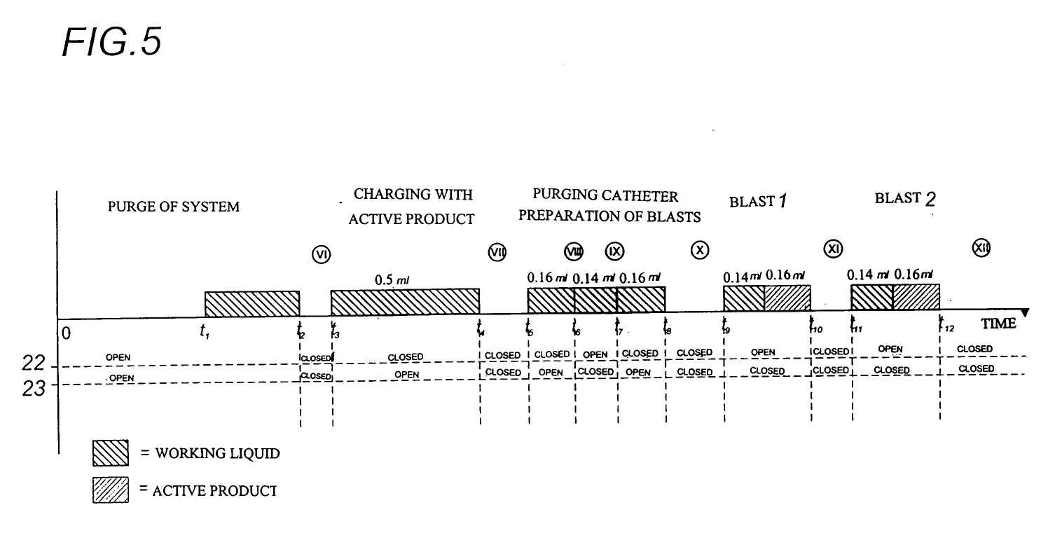

FIG. 5 is a graph showing the nature and quantity of liquid delivered by the apparatus over the course of time, during a particular exemplary application of two successive microscopic injections of active product; and

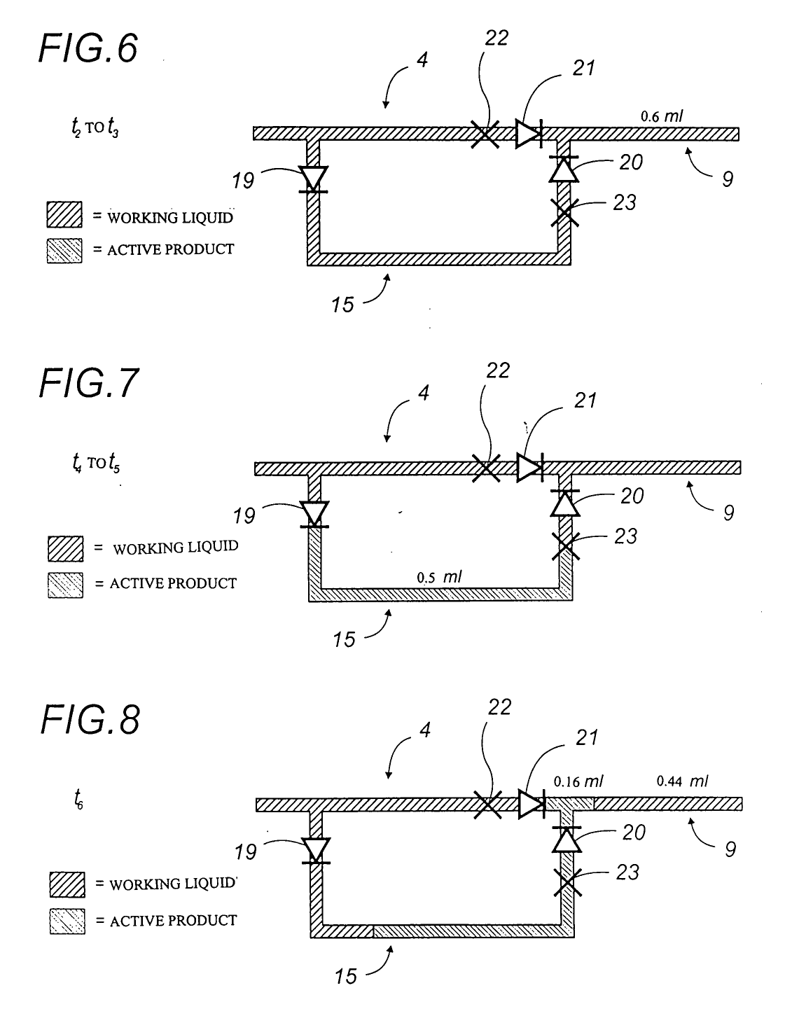

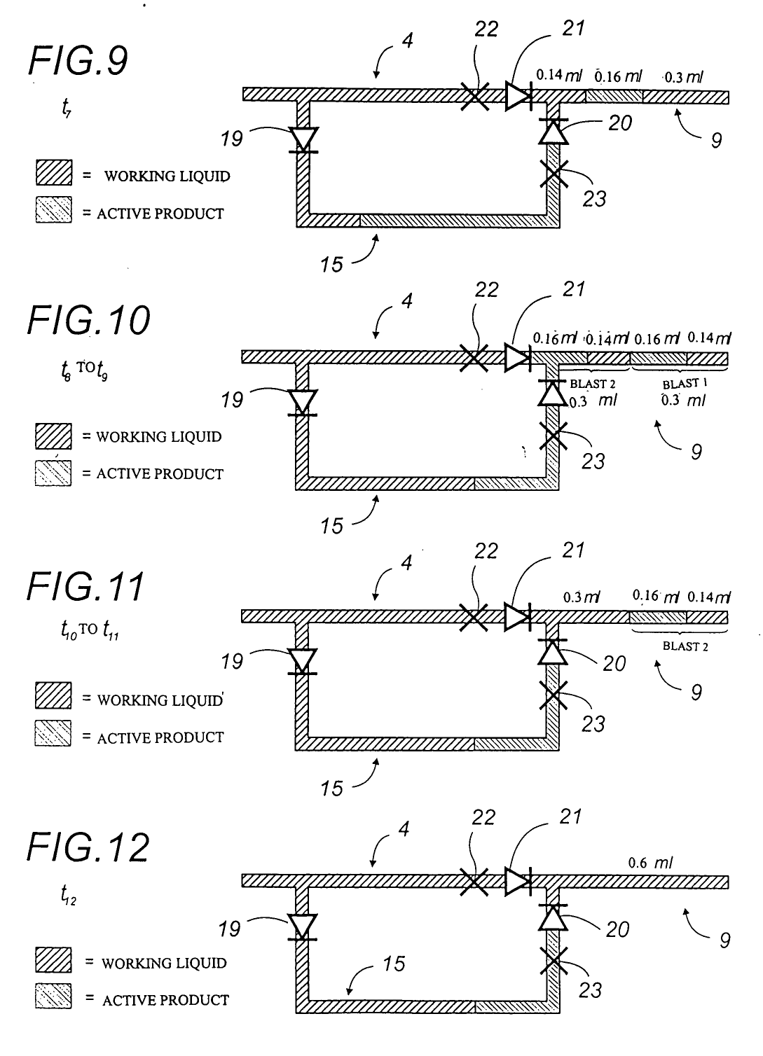

FIGS. 6 through 12 are schematic views of one portion of the injection apparatus according to the invention that corresponds to the principal branch and the derivation branch of the circuit and to the catheter, in various configurations corresponding to different moments referenced from VI to XII on the graph in FIG. 5, throughout the example of application shown in FIG. 5.

The injection device of the present invention will now be described in detail with reference to FIGS. 1 through 12. Equivalent elements shown in different drawings will bear the same reference numerals.

FIG. 1 is a schematic representation of an injection apparatus 1 according to the invention capable of sending one or more jets of sterile pressurized working liquid and injecting one or more microscopic quantities of active product.

This apparatus comprises a reserve 2 of working liquid, for example a flexible plastic pouch containing the working liquid connected to a generator 3 of pressurized liquid.

According to a preferred embodiment, pressurized liquid generator 3 is an enclosure surrounding the reserve 2 of working liquid which is filled with neutral pressurized gas in order to compress the pouch and pressurize the liquid.

The pressure of the liquid jet generated can be regulated according to need. The interior pressure of the enclosure preferably ranges from 0.3 to 200 bars and is preferably between 0.3 and 100 bars.

The enclosure can also be thermostatically heated to a temperature near or slightly higher than 37° C. so the working liquid reaching the intervention site will be approximately the same temperature as the patient's body.

The working liquid used is preferably sterile physiological serum pressurized according to the application. One high pressure value adapted for the working liquid, cited here only by way of example, is approximately 20 bars.

Obviously other sterile liquids can be used as the sterile working liquid such as, for example, saline solution, glucose solution, Ringer-lactate, hydroxy-ethyl-amidin or a mixture of these solutions.

The sterile working liquid flows through the tube forming the principal branch 4 of the hydraulic circuit to a handpiece 5 that allows the user to control the release of the jet of working liquid and/or active product and direct it.

Handpiece 5 comprises an ergonomic body for easy gripping and manipulation which may have controls such as pushbuttons 7.

Handpiece 5 extends into active extremity 8. In this variation it is a catheter 9. The catheter is a conventional flexible conduit 10 through which and along which the pressurized sterile working liquid, as well as the active or treatment product, move forward.

Catheter 9 comprises a proximate extremity 11 manipulated by the surgeon and a distal extremity 12 introduced inside the patient's body.

The distal extremity 12 of the catheter has one or more orifices through which the working liquid and the active product are ejected.

Catheter 9 may comprise known means of navigation and orientation, which are not shown in the drawings because they do not constitute part of the invention. Its distal extremity 12 is articulated and may further comprise an indexing system for positioning it in the proper location and at the preferred angle, with the entire unit being controlled by handpiece 5.

Catheter 9 may also comprise an anchor system for immobilizing the active distal extremity while liquid is being blasted, thereby making the intervention more precise. This may consist of any mechanical anchoring means or a device using suction.

In this case injection apparatus 1 comprises a suction system 13 connected to a vacuum source 14, for example, a vacuum pump or the hospital's general vacuum circuit. These optional elements are shown by broken lines in FIG. 1.

It is also possible for a variation of catheter 9 to comprise a suction conduit concentrically surrounding conduit 10 and flaring out near distal extremity 12 of the catheter to form a skirt.

Then, having positioned distal extremity 12 of the catheter beside the area of tissue or the wall of the organ to be treated, the surgeon can place the distal extremity on that area by engaging the suction near the skirt, which also serves as a nozzle.

Advantageously, the skirt physically isolates the injection site from the flow of blood and from the rest of the patient's body.

The active product, which is useful and beneficial in the targeted zone, actually may be harmful or even toxic if applied to other organs of the patient that are not involved in treatment. This is particularly true during gene therapy, in which genes, cells, nucleic acids, or genetically modified proteins may be administered to the target organ in isolation or using a vector, for example, a virus.

Thus, it is very important that the administered product, developed for a specific application, remain localized at the injection site and not be carried beyond the treatment site by blood circulation in order to avoid the misfortune of having it end up in other non-targeted organs.

Using the apparatus of the invention the active product is injected deeply into the core of the tissue to be treated, strongly decreasing the possibility that it would escape from the injection channel and end up in an organ not involved in the treatment.

If distal extremity 12 of the catheter is equipped with a suction skirt, the injection site is completely isolated from the patient's blood circulation until the active product has diffused completely throughout the tissue concerned. Therefore, the risk of accidental dispersion is diminished ever further.

To further decrease risk, these active principles are often formulated in particularly viscous liquid in order to prevent it from flowing outside the injection channel. Advantageously, the apparatus of the invention adapts perfectly to injecting products of any viscosity.

According to a preferred embodiment not shown, catheter 9 may comprise at its distal extremity a retractable perforating or puncturing tool, preferably a retractable type needle, moving between an extended working position and a retracted safety position inside the catheter for the purpose of creating the puncture that initiates formation of the hollow injection channel by mechanical perforation.

According to a previously patented innovation the tool at the extremity extends automatically under pressure from liquid exiting from the catheter, and the tool at the extremity automatically retracts inside the catheter in the absence of liquid or if the pressure of the liquid drops below a threshold value.

Injection apparatus 1 also comprises a secondary derivation branch 15 for containing the active product to be injected.

This secondary tube 15 is preferably connected to a reserve of active product or treatment product 16 by a connecting device 17.

The reserve of active product 16 may be any type. It may consist of a system for automatically discharging a precise dose of active product. It may also consist of a simple syringe in which the surgeon has placed the active product and which he uses to introduce the active product into the injection apparatus through the connecting device 17.

Connecting device 17 may consist, for example, of a triple track charge valve 18 as shown in the drawings. In order to avoid mistakes during manipulation, charge valve 18 preferably is a two-track valve.

By simply opening valve 18 the surgeon can first of all place the reserve of active product 16 in communication with secondary branch 15 of the apparatus in order to charge it with active product. The charge valve is then placed in the configuration shown in FIG. 3. The surgeon can then fill secondary tube 15 with the contents of a syringe.

Next, he can place secondary derivation branch 15 in communication with principal branch 4, and the apparatus is then ready for injection. The tap is then in the configuration of FIGS. 1 and 2.

A person skilled in the art can easily conceive of a variation of the apparatus of the invention where these steps would take place automatically.

Secondary derivation branch 15 is isolated from principal branch 4 of the fluid circuit by two anti-return flow control devices 19 and 20 positioned at the two extremities of the derivation tube, at its inlet and outlet, respectively.

An anti-return flow control device 21 is preferably disposed at the outlet of the principal tube 4 to prevent any working liquid or active product from flowing back and causing contamination in the upstream portion of the fluid circuit.

The circulation of fluid in each of these branches of the circuit depends upon whether the two isolation valves 22 and 23 provided on the main branch and the secondary branch of the circuit, respectively, are open or closed.

According to a preferred embodiment, at least one of these isolation valves 22 or 23, and preferably both, comprises a cam-shaped roller which, when closed, crushes the tube from the outside.

For technical reasons related to the nature of the conduits, both isolation valves 22 and 23 are attached in the immediate vicinity of anti-return flow control devices 20 and 21. If technical or technological conditions permit, anti-return flow control devices 20 and 21 are integrated with isolation valves 22 or 23, and vice versa.

According to an advantageous mode, these two valves are controlled, preferably electronically, by a multiplexing means 24. According to an essential feature of the invention, these two isolation valves 22 and 23 are activated independently of each other.

Multiplexer 24 controls the opening and closing of isolation valves 22 and 23 as a function of whatever parameters of time and duration have been previously stored by the surgeon in order to achieve the optimum injection sequence for the particular case to be treated.

It is possible to modify the opening time of isolation valves 22 and 23 as a function of the viscosity of the liquids whose passage through them is being controlled.

When isolation valve 22 is open and isolation valve 23 is closed, a jet of pressurized working liquid is ejected from distal extremity 12 of catheter 9.

This blast of pressurized working liquid may be used to form a hollow injection channel, for example. It may also be used for any other suitable cutting or dissection application, either prior to or following injection.

To improve effectiveness, the blast of pressurized working liquid may be a pulsating blast achieved using a method known in this domain.

When isolation valve 22 is closed and isolation valve 23 is open, the pressurized working liquid circulates in derivation branch 15 and pushes against the active product present in the secondary branch. In this way the active product is propelled by the pressurized working liquid to the distal extremity of the catheter and it is injected into the previously hollowed-out injection conduit.

If the two isolation valves are open simultaneously, a mixture forms at the junction of the two branches of the circuit and diluted active product is ejected at the catheter outlet.

As mentioned in the introductory portion of this application, numerous injection possibilities become available to the surgeon when the parameters for opening and closing isolation valves 22 and 23 are varied.

The injection apparatus according to the invention ensures the total sterility required for surgical interventions. Actually, all the elements of the fluid circuit that are capable of becoming contaminated are disposable, single-use sterile elements: from the sterile pouch holding the working liquid to the reserve of active product, the different tubes, flow devices, valves, connectors and other hydraulic elements, as well as the handpiece and the catheter.

In FIG. 1, all the disposable elements of the injection apparatus designed for a single use are shown in gray. The elements designed to be saved are shown in black.

FIG. 4 shows in more detail another embodiment of the invention. According to this variation, the principal branch 4 and secondary branch 15 of the circuit are disposed in parallel and connected at their extremities by bifurcations 25 and 26.

Principal branch 4 leads respectively, from bifurcation 25 and in the direction of fluid flow, into a tube 27 that can be flattened, preferably at its extremity, by the cam on isolation valve 22, an anti-return flow control device 21, and a tube 28 terminating at bifurcation 26.

After bifurcation 25 a tube 32 common to both branches supplies handpiece 5.

According to the variation in FIG. 4, it is possible for the injection apparatus to have no valve charging it with active liquid. In this embodiment, tube 30 assumes the direct role of active product reserve 16. A portion of tube 30 serves as a calibrated reserve of a precise dose of active product.

It may vary in length and diameter according to the volume of active product to be injected. Such a system advantageously permits the injection of very small quantities of active product. It is also possible, by adapting the length of tube 30, to obtain a measured reserve of 1 or 2 ml of active product.

This feature responds perfectly to the concerns encountered in the field of gene or cell therapy during which very small amounts of active product are generally injected. Because of the small volume, conventional systems become problematic and the usual containers, such as flexible plastic pouches, prove to be unsuitable.

The apparatus of the invention provides a simple and effective way to inject micro-quantities of active product in precise doses.

However, when the active product contains live cells, it is important to use tubes with a sufficient diameter. Conduits that are too narrow actually cause cell death, rendering the injection completely ineffective.

According to a preferred embodiment, a suitable interior diameter for the tubes is approximately 1.5 mm.

In order to decrease the volume of the reserve of active product while still conserving it, it is possible to decrease the length of calibrated tube 30.

The tubes, anti-return flow control devices, valves, and other elements on the fluid circuit are made of material resistant to high pressure. For example, high pressure tubes made of braided polyamide may be used.

Furthermore, certain active products are sensitive to high pressure and cannot be injected under the same pressure as the working liquid without the risk of deterioration and reduced effectiveness.

In this case, according to a variation of the invention shown in FIG. 3, secondary derivation branch 15 may comprise a tubular portion 33 with a reduced interior diameter used to substantially reduce the pressure of the liquid passing through it.

This reduced diameter tubular portion 33 preferably replaces the portion of tube 29 situated at the inlet of the secondary derivation branch 15 before anti-return flow control device 19.

When isolation valve 23 is open, the working liquid under pressure coming from reserve 2 and pressurized liquid generator 3 enters the tubular portion or restriction 33 having the extremely reduced diameter. This narrowing causes a considerable decrease in its pressure before it contacts the active product present in tube 30 of the derivation branch.

Therefore, the active product can be ejected with considerably lower pressure than that of the working liquid, advantageously preserving the more fragile substances.

This decrease in pressure can be modulated by playing with the diameter and length of restriction 33.

According to a preferred variation the tube or restriction 33 may have a interior diameter of approximately 0.3 mm.

Since only the working liquid flows through tubular portion 33, it is possible to decrease tube diameter significantly without risking the deterioration of live cells of active product.

Thus, according to an essential feature of the invention, the injection apparatus advantageously makes it possible to form an injection channel under high pressure and then inject an active product at low pressure.

Furthermore, the use of a reduced or restricted diameter tubular portion 33 also allows micro-quantities of active product to be injected.

Actually, for technical reasons, the opening and closing time of isolation valve 23 does not need to be decreased beyond the minimal duration limit imposed by the characteristics of the apparatus. This minimal opening duration causes injection of a minimal quantity of active product that cannot be reduced at the same injection pressure.

By decreasing injection pressure using restriction 33 it is possible to reduce the quantity of active produced injected with isolation valve 23 remaining open for the same length of time.

The apparatus of the invention also advantageously allows injection of micro-quantities of active product.

The operation of the apparatus of the invention will be more understandable from the description of a precise example of its operation corresponding to FIGS. 5 through 12. However, it should be noted that this is merely one particular instance showing how the apparatus of the invention is used, the goal being to illustrate one of the numerous possible applications offered by the injection apparatus and not to limit protection in any way.

This example illustrates an injection constituting two successive blasts of 160 μl of active product, each blast being preceded by a blast of 140 μl of working liquid under pressure in order to form the hollow injection channel.

A reserve of active product is used, for example, 500 μl.

The injection apparatus used comprises a catheter with a volume of 600 μl and a derivation tube calibrated at 500 μl.

In the various drawings the working liquid is symbolized by black hatching and the active product by reverse gray hatching.

At the outset the surgeon begins to purge the injection apparatus of any air it contains in order to fill it with working liquid. To do this, he or she opens isolation valves 22 and 23 and engages the flow of working liquid.

Charge valve 18 must be placed in the configuration shown in FIGS. 1 and 2, allowing communication between secondary branch 15 and principal branch 4.

Little by little the working liquid fills principal branch 4, derivation branch 15, and catheter 9. At time t₁, some working liquid begins to flow through the distal extremity of the catheter.

At time t₂ the surgeon closes isolation valves 22 and 23. The flow of working liquid at the catheter outlet ceases. The entire internal hydraulic circuit of the injection apparatus is filled with working liquid. The apparatus is then in the configuration shown in FIG. 6.

Next the surgeon fills the apparatus with active product. To do this he opens charge valve 18, as in FIG. 3, causing derivation conduit 15 to communicate with the reserve of active product 16. This may be done by placing a syringe filled with active product the at inlet of valve 18.

At time t₃ he opens valve 23 and begins to fill conduit 15 with active product. This forces out the working liquid initially present. A quantity of working liquid equivalent to the quantity of active product introduced flows out of the catheter. In the example shown this quantity is 500 μl.

At time t₄ the introduction of active product is terminated. The surgeon closes isolation valve 23 and replaces charge valve 18 in the position shown in FIG. 2, causing the two branches of the circuit to again communicate. The apparatus is then in the configuration shown in FIG. 7.

Next, the blasts are prepared by successively disposing the packets of liquid in the appropriate order and quantity and by placing them in the blasting position at the extremity of the catheter just before the outlet orifice.

To do this, isolation valve 23 is opened at time t₅ for a duration corresponding to the time it takes for 160 μl of active product, pushed by the flow of 160 μl of pressurized working liquid, to flow through this valve into derivation branch 15.

A corresponding quantity of working liquid escapes through the outlet of catheter 9.

The apparatus is then in the configuration shown in FIG. 8.

At time t₆ isolation valve 23 is closed and isolation valve 22 is open until time t₇. The duration for which the valve is open corresponds to the time it takes for 140 μl of pressurized working liquid to flow through valve 22. 140 μl of working liquid is simultaneously expelled from the catheter.

At time t₇ isolation valve 22 is closed and isolation valve 23 is open until time t₈. The duration for which the valve is open corresponds to the time it takes for 160 μl of active product to flow through valve 23, pushed by the pressurized working liquid. 160 μl of working liquid is simultaneously expelled from the catheter.

At time t₈ the two isolation valves are closed so as to block flow. The apparatus is then in the situation shown in FIG. 10. It is ready to perform the injections.

The surgeon places the active distal extremity of the catheter, in blasting position, into contact with the area of the organ wall or tissue to be treated.

At time t₉ he engages the first blast. Isolation valve 22 is open from t₉ to t₁₀, which corresponds to the injection duration of the first blast from the distal extremity of the catheter. This blast is composed of a first impulse of 140 μl of pressurized working liquid, which hollows out the injection channel, followed by a second impulse of 160 μl of active product injected into the channel during the second blast.

At time t₁₀ the two valves are closed. The apparatus is then in the situation shown in FIG. 11. The surgeon can displace the active extremity of the catheter into the second blasting position.

At time t₁₁ he releases the second blast. Valve 22 is open from t₁₁ to t₁₂, corresponding to the time necessary for ejecting the second blast from the distal extremity of the catheter. This blast, like the preceding one, is composed of a first impulse of 140 μl of pressurized working liquid used to hollow out the injection channel, followed by a second impulse of 160 μl of active product injected into the channel during the second blast.

At time t,₁₂ the two valves are closed. The apparatus is then in the configuration shown in FIG. 12.

The two blasts may possibly take place in succession at the same location. In this case, the time interval t₁₀-t₁₁ during which the two isolation valves are closed becomes superfluous and can be eliminated, with the two blasts proceeding in succession without interruption.

According to a feature of the invention, the opening time of isolation valves 22 and 23 is modified as a function of the viscosity of the liquids used.

To summarize, the method of generating a sequence of liquid jets according to the invention may comprise the following steps:
purging the apparatus to expel the air inside in order to fill it with working liquid;
charging the apparatus with active product;
preparing one or two blasts by successively disposing packets of liquid in the appropriate order and quantity and placing them in blasting position at the active distal extremity of the apparatus just before the outlet orifice;
placing the active distal extremity of the apparatus in blasting position;
effecting at least one blast of a sequence of liquid jets.

The apparatus of the invention is useful for a wide variety of medical, surgical, or diagnostic interventions on any organ of a living being or in surgery ex-situ.

For example, the surgical apparatus of the invention is particularly well-adapted for use in interventions such as transmyocardial or myocardiac revascularization, because it allows the formation of revascularization conduits in the ischemic areas of the myocardium and subsequent injection through these conduits of angiogenic substances, for example, growth factor.

The apparatus of the invention allows injection of diverse substances such as, for example, therapeutic products, products relating to an intervention in progress or diagnostic or contrast products. They may be conventional chemical molecules or products from biotechnology and genetics.

Obviously, the invention is not limited to an apparatus capable of injecting only one active product. It is possible to conceive, in accordance with the principles of the invention, an injection apparatus comprising several derivation branches, disposed in series or in parallel, for application of various active products either as a mixture or in succession, according to previously programmed parameters.

Likewise, the apparatus and method previously described are not limited to applications in the surgical, medical, or diagnostic domain. The method of generating a sequence of jets of liquid according to the invention is a general method which could have applications in numerous other domains such as, for example, construction, agriculture, the food industry, chemistry or biology laboratories, printing and graphics, tattooing, cleaning, and so forth.

The following is a non-exhaustive list of examples of possible uses for the apparatus and/or the method of the invention:
in construction for treating wood, injecting hardening agents, treating or drying walls and partitions;
in laboratories for microscopically injecting reagents into base products designed to be analyzed or used in preparing vaccines or medications;
in the agriculture-food industry, especially for injecting flavor, taste, preservative, colorant, or the like;
for injecting coloring products such as ink into human or animal skin for decorative or identifying tattoos, or onto other types of support for purposes of marking, drawing, or printing.

1-29. (canceled)

30. An apparatus for injecting an active product comprising a reserve (2) of working liquid pressurized by a pressurized liquid generator (3), a handpiece (5) terminating in an active extremity (8) that comprises an outlet means for a pressurized jet of working liquid and a jet of active product wherein,
a principal fluid circuit branch (4) connecting a reserve (2) of working liquid to the handpiece (5), with flow of liquid through the principal fluid circuit branch being controlled by one of an opening or closing of a first isolation valve (22);
a secondary derivation branch (15) parallel to the principal fluid circuit branch (4) and fluidly isolated from the principal fluid circuit branch (4), designed to contain active product, with flow through the principal fluid circuit branch (4) being controlled by one of an opening or closing of a second isolation valve (23); and
a multiplexing means (24) for independent control of the one of the opening and closing of the first and second isolation valves (22, 23) according to predetermined parameters:

31. The apparatus according to claim 30 wherein the apparatus further comprises a multiplexing means (24) for independent control over the opening and closing of the isolation valves (22, 23) according to any predetermined parameters previously selected and stored by a surgeon as a function of the particular case to be treated.

32. The apparatus according to claim 30 wherein the reserve (2) of working liquid is a flexible plastic pouch containing the working liquid and the generator (3) of pressurized liquid is an enclosure surrounding the reserve (2) of working liquid which is filled with neutral gas until the reserve (2) is pressurized sufficiently to compress the pouch and pressurize the liquid.

33. The apparatus according to claim 30 wherein the active extremity (8) is a catheter (9).

34. The apparatus according to claim 33 wherein the catheter (9) comprises at a distal extremity a retractable perforating or puncturing tool movable between an extended working position and a retracted safety position inside the catheter.

35. The apparatus according to claim 34 wherein the catheter (9) has at the distal extremity a retractable perforating or puncturing tool that is movable and has a retractable needle.

36. The apparatus according to claim 30 wherein the apparatus further comprises a suction system (13) connected to a vacuum source (14).

37. The apparatus according to claim 30 wherein an anti-return flow control device (21) is located at an outlet of the principal branch (4).

38. The apparatus according to claim 30 wherein the secondary derivation branch (15) is isolated from the principal branch (4) by means of two anti-return flow control devices (19, 20) attached to two extremities of a derivation tube.

39. The apparatus according to claim 30 wherein the first and second isolation valves (22, 23) are attached at an immediate proximity, respectively, of anti-return flow control devices (21) and (20).

40. The apparatus according to claim 39 wherein the anti-return flow control devices (20) and (21) are integrated within the second and first isolation valves (23) and (22), respectively.

41. The apparatus according to claim 30 wherein the secondary derivation branch (15) is connected to a reserve of active product (16) by a communicating device (17).

42. The apparatus according to claim 41 wherein the communicating device (17) is a charge valve (18) with two or three tracks.

43. The apparatus according to claim 30 wherein the derivation branch (15) comprises a tubular portion (30) serving as a reserve that is calibrated for one precise dose of active product.

44. The apparatus according to claim 30 wherein the secondary derivation branch (15) comprises a tubular portion or restriction (33) having a reduced interior diameter, used to break the pressure of the liquid passing through it.

45. The apparatus according to claim 44 wherein the tubular portion or restriction (33) of reduced interior diameter is located at an inlet to the secondary derivation branch (15) before the anti-return flow control device (19).

46. The apparatus according to claim 30 wherein at least one of the first and second valves (22, 23) on the two circuit branches comprises a cam-shaped roller which, when closed, crushes the tube from the outside.

47. The apparatus according to claim 30 wherein the elements on the fluid circuit that could become contaminated are sterile, disposable, single-use elements.

48. The injection method using the injection apparatus according to claim 30 wherein a hollow injection channel is formed by blasting a jet of pressurized working liquid, and then in the next step the active product is injected through the injection channel.

49. A method of generating a sequence of liquid jets using an apparatus comprising a reserve (2) of working liquid pressurized by a pressurized liquid generator (3), a handpiece (5) terminating in an active extremity (8) comprising outlet means for a jet of pressurized working liquid and a jet of active product, a principal fluid circuit branch (4) connecting the reserve of working liquid (2) to the handpiece (5), with the flow of liquid through the branch being controlled by opening or closing an first isolation valve (22), a secondary derivation branch (15) parallel to the principal branch (4) and fluidly isolated from the principal branch, designed to contain an active product, with the flow of liquid through the secondary derivation branch (15) being controlled by opening or closing second an isolation valve (23), and a multiplexing means (24) for independent control over the opening and closing of the isolation valves (22, 23) according to predetermined parameters, characterized by generating a sequence consisting of at least one impulse of pressurized working liquid followed by a microscopically dosed impulse of active product.

50. The method of generating a sequence of liquid jets according to claim 49 wherein the first isolation valve (22) on the principal branch (4) on the circuit is opened to generate an impulse formed of an appropriate quantity of pressurized working liquid, and then in a later phase the second isolation valve (23) on the derivation branch (15) is opened to generate an impulse formed of the desired quantity of active product.

51. The method of generating a sequence of liquid jets according to claim 49 wherein the impulse of active product is generated at a pressure which essentially corresponds to that of the impulse of working liquid.

52. The method of generating a sequence of liquid jets according to claim 49 wherein the impulse of active product is generated at less pressure than that of the working liquid and preferably at low pressure.

53. The method of generating a sequence of liquid jets according to claim 49 wherein the impulse of active product is followed by another impulse of pressurized working liquid.

54. The method of generating a sequence of liquid jets according to claim 49 wherein the quantities of liquid forming the impulses of working liquid and active product are precisely established by fixing the opening time of each of the first and second isolation valves (22, 23).

55. The method of generating a sequence of liquid jets according to claim 54 wherein the opening time of the first and second isolation valves (22, 23) varies as a function of viscosity of the liquids.

56. A method of generating a sequence of liquid jets using an apparatus comprising a reserve (2) of working liquid pressurized by a pressurized liquid generator (3), a handpiece (5) terminating in an active extremity (8) comprising outlet means for a pressurized jet of working liquid and a jet of active product, a principal fluid circuit branch (4) connecting the reserve of working liquid (2) to the handpiece (5), with the flow of liquid through the principal fluid circuit branch (4) being controlled by the opening or closing of a first isolation valve (22), a secondary derivation branch (15) parallel to the principal fluid circuit branch (4) and fluidly isolated from the principal fluid circuit branch (4), designed to contain active product, with the flow of liquid through the secondary derivation branch (15) being controlled by the opening or closing of a second isolation valve (23), and a multiplexing means (24) to independently control the opening and closing of the first and second isolation valves (22, 23) according to predetermined parameters, wherein the first and second isolation valves (22, 23) of the two branches (4, 15) of the circuit are opened simultaneously in order to generate a jet consisting of a mixture of active product and working liquid in proportions that are precisely fixed by regulating the opening time of each of the first and second isolation valves (22, 23).

57. The method of generating a sequence of liquid jets according to claim 49 wherein a very small quantity of active product is used and the active product is used in microscopic doses.

58. The method of generating a sequence of liquid jets according to claim 49 further comprising the steps of:
purging air from the apparatus in order to fill it with working liquid;
charging the apparatus with active product;
preparing one or more blasts by successively disposing packets of fluid in the appropriate order and quantity and placing them in blasting position at the active distal extremity of the apparatus just before the outlet orifice;
placing the active distal extremity of the apparatus in blasting position; and
performing at least one blast of a series of liquid jets.
